(19) United States
(12) Patent Application Publication
(10) Pub. No.: US 2023/0324266 A1
(43) Pub. Date: Oct. 12, 2023

(54) PURIFICATION OF PRION PROTEIN SUBSTRATE FOR REAL-TIME QUAKING INDUCED CONVERSION (RT-QUIC)

(71) Applicant: Davin Henderson, Fort Collins, CO (US)

(72) Inventor: Davin Henderson, Fort Collins, CO (US)

(21) Appl. No.: 18/193,872

(22) Filed: Mar. 31, 2023

Related U.S. Application Data
(60) Provisional application No. 63/326,053, filed on Mar. 31, 2022.

(51) Int. Cl. G01N 1/34

(52) U.S. Cl. CPC G01N 1/34; G01N 2001/4088

(57) ABSTRACT

A method for purifying protein substrates for Real-Time Quaking Induced Conversion (“RT-QuIC”) is provided that can efficiently yield purified protein substrates desirable for RT-QuIC testing. By modifying certain parameters of the protein solubilization, the purification receptacle, the dialysis treatment, and the storage conditions, the purification and production of prion protein amplification substrate useful for RT-QuIC testing can be optimized.

### RELATED APPLICATIONS

This application claims the priority benefit under 35 U.S.C. § 119(e) of U.S. Provisional Pat. Application Serial No. 63/326,053 entitled “PURIFICATION OF PRION PROTEIN SUBSTRATE FOR REAL-TIME QUAKING INDUCED CONVERSION (RT-QUIC),” filed Mar. 31, 2022, the entire disclosure of which is incorporated herein by reference.

### BACKGROUND

### 1. Field of the Invention

The present disclosure generally relates to the purification of protein substrates for Real-Time Quaking Induced Conversion (“RT-QuIC”).

### 2. Description of the Related Art

Prion diseases are fatal neurodegenerative disorders that are caused and spread via abnormal prion proteins. An example of a prion disease is chronic wasting disease (“CWD”). The infectious agent, or prion, of prion diseases appears to be composed primarily of an abnormal, misfolded, oligomeric form of a protein (i.e., a “prion”), which are formed post-translationally from normal cellular proteins. These abnormal prions, which in purified form can resemble amyloid fibrils, may induce the polymerization and conformational conversion of normal cellular proteins to infectious abnormal prion proteins. Thus, these abnormal prions may self-propagate in the form of seeded or templated polymerization.

In order to address the deficiencies of previous CWD diagnostic methods, CWD testing by using real-time quaking induced conversion (“RT-QuIC”) may be used. The RT-QuIC test combines features of quaking-induced conversion (QuIC) and amyloid seeding assay (ASA) methods and involves prion-seeded conversion of the alpha helix-rich form of bacterially-expressed recombinant cellular protein to a beta sheet-rich amyloid fibrillar form. The RT-QuIC test is as sensitive as an animal bioassay but can be accomplished in two days or less.

Generally, the RT-QuIC test utilizes a prion protein amplification substrate, which is used for the amplification of the test prion for RT-QuIC testing. Thus, in order to carry out RT-QuIC testing, an adequate supply of prion protein amplification substrate must be readily available. However, processes for producing and purifying prion protein amplification substrates currently exhibit one or more deficiencies that limit the efficient production of this valuable substrate. Therefore, an improved method for producing and purifying prion protein amplification substrates is needed.

### SUMMARY

One or more embodiments concern a method for extracting and purifying a prion protein amplification substrate for RT-QuIC testing. Generally, the method comprises: (a) providing a protein aggregate derived from an initial quantity of a bacterial expression; (b) contacting at least a portion of the protein aggregate with at least 20 mL of at least one protein denaturant for at least one hour to thereby form a solubilized protein aggregate; (c) contacting at least a portion of the solubilized protein aggregate with at least one protein affinity resin to thereby form a resin mixture; (d) introducing at least a portion of the resin mixture into at least one purification receptacle, wherein the purification receptacle has a maximum transverse interior dimension and a maximum longitudinal interior dimension; and (e) purifying at least a portion of the resin mixture to thereby form a purified protein amplification substrate. Furthermore, (i) the maximum transverse interior dimension is less than 25 mm and the ratio of the maximum longitudinal interior dimension to the maximum transverse interior dimension is at least 27:1, when the resin mixture introduced into the purification receptacle comprises less than 30 mL of the solubilized protein aggregate; and (ii) the maximum transverse interior dimension is at least 25 mm and the ratio of the maximum longitudinal interior dimension to the maximum transverse interior dimension is less than 27:1, when the resin mixture introduced into the purification receptacle comprises at least 30 mL of the solubilized protein aggregate.

One or more embodiments concern a method for extracting and purifying a prion protein amplification substrate for RT-QuIC testing. Generally, the method comprises: (a) providing a protein aggregate derived from an initial quantity of a bacterial expression; (b) contacting at least a portion of the protein aggregate with at least one protein denaturant for at least eight hours to thereby form a solubilized protein aggregate; (c) contacting at least a portion of the solubilized protein aggregate with at least one protein affinity resin to thereby form a resin mixture; (d) introducing at least a portion of the resin mixture into at least one purification receptacle; (e) purifying at least a portion of the resin mixture to thereby form a purified protein amplification substrate; and (f) storing at least a portion of the purified protein amplification substrate at a temperature greater than 0° C. and less than 10° C. for at least one hour to thereby form the prion protein amplification substrate.

One or more embodiments concern a method for extracting and purifying a prion protein amplification substrate for RT-QuIC testing. Generally, the method comprises: (a) providing a protein aggregate derived from an initial quantity of a bacterial expression; (b) contacting at least a portion of the protein aggregate with at least one protein denaturant for at least nine hours to thereby form a solubilized protein aggregate; (c) contacting at least a portion of the solubilized protein aggregate with at least one protein affinity resin to thereby form a resin mixture; (d) introducing at least a portion of the resin mixture into at least one purification column; (e) subjecting at least a portion of the resin mixture to Fast Protein Liquid Chromatography (FPLC) to thereby form an eluted protein fraction and a spent affinity resin; (f) contacting at least a portion of the eluted protein fraction with at least 2.5 mL of dialysis buffer to thereby form a dialyzed prion protein fraction; and (g) storing at least a portion of the dialyzed protein fraction at a temperature of at least 1° C. and less than 10° C. for at least one hour to thereby form the purified protein amplification substrate.

### BRIEF DESCRIPTION OF THE FIGURES

Embodiments of the present invention are described herein with reference to the following drawing figures, wherein:

FIG. 1 depicts an exemplary purification column that may be used in accordance with one or more embodiments.

### DETAILED DESCRIPTION

We have discovered a superior method for extracting and purifying a prion protein amplification substrate for RT-QuIC testing. More particularly, we have discovered that by optimizing certain parameters of the solubilizing step, the purification receptacle, the dialysis step, and the storage step, we can optimize the purification and production of prion protein amplification substrate useful for RT-QuIC testing. The new and inventive protocol is provided below and further described in the accompanying claims.

As discussed below in greater detail, the inventive method for extracting and purifying a prion protein amplification substrate for RT-QuIC testing generally comprises: (a) providing a protein aggregate derived from an initial quantity of a bacterial expression; (b) contacting at least a portion of the protein aggregate with at least one protein denaturant for at least one hour to thereby form a solubilized protein aggregate; (c) contacting at least a portion of the solubilized protein aggregate with at least one protein affinity resin to thereby form a resin mixture; (d) introducing at least a portion of the resin mixture into at least one purification receptacle, wherein the purification receptacle has a maximum transverse interior dimension and a maximum longitudinal interior dimension; and (e) purifying at least a portion of the resin mixture to thereby form a purified protein amplification substrate. Furthermore, in various embodiments, (i) the maximum transverse interior dimension is less than 25 mm and the ratio of the maximum longitudinal interior dimension to the maximum transverse interior dimension is at least 27:1, when the bacterial expression introduced into the purification receptacle contains less than 13 grams of cells, and (ii) the maximum transverse interior dimension is at least 25 mm and the ratio of the maximum longitudinal interior dimension to the maximum transverse interior dimension is less than 27:1, when the bacterial expression introduced into the purification receptacle contains at least 13 grams of cells. Each of these steps is discussed and described in greater detail below.

In the first step of the method, a protein aggregate may be provided that is derived from an initial quantity of a bacterial expression. As used herein, a “bacterial expression” refers to the post-expression bacterial cell culture (before lysing) used to form the protein aggregate. Protein expression in bacteria is fairly straightforward: DNA coding the protein of interest is inserted into a plasmid expression vector, which is then transformed into a bacterial cell. The transformed cells propagate and are induced to produce the protein of interest, thereby forming a “bacterial expression” comprising high amounts of the protein of interest. The bacterial cells are then lysed to release the produced protein of interest. The protein can then be purified from the cellular debris to form the protein aggregate (also referred herein as an “inclusion body”).

The bacterial cellular contents of the bacterial expression can be important and may dictate what reaction conditions and purification receptacle parameters are utilized during the purification method described herein. Generally, the bacterial expression (prior to lysing) may comprise the plasmid-modified bacterial cells, which contain the amplified protein of interest. The bacterial expression may also contain an expression liquid substrate, in which the protein expression conditions are optimized for the production of the protein of interest by the bacterial cells.

In one or more embodiments, the bacterial expression has an optical density at 600 nm of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ODU. Additionally, or in the alternative, the bacterial expression has an optical density at 600 nm of less than 70, 65, 60, 55, 50, 45, 40, 35, 30, 25, 20, 15, or 10 ODU. The optical density may be measured using a conventional spectrometer known in the art.

In one or more embodiments, the recombinant protein of interest being amplified in the bacterial cells (and within the bacterial expression) may be derived from any protein source, including from the same species as the source of the intended test subject (e.g., whitetail deer) or from a different species relative to the test subject. Exemplary sources of the recombinant protein of interest can include bovine, ovine, hamster, rat, mouse, canine, feline, cervid, human, or non-human primate protein substrates.

In one or more embodiments, recombinant Syrian hamster PrP protein (aa 90-231) (“SHrPrP”) may be utilized as the protein of interest when forming the bacterial expression and the designated bacterial cell may be Escherichia coli, such as BL21 DE3 Escherichia coli. The SHrPrP may be inserted into bacterial cells via a plasmid. Overnight Express Auto Induction from EMD Biosciences may be used to induce expression for about 24 hours (in accordance with the established protocol). Subsequently, the Bug Buster reagent (EMD Biosciences) and Lysonase (EMD Biosciences) may be used for cell lysis, and protein aggregate (i.e., inclusion body) isolation may be accomplished via the protocol included with the Bug Buster reagent (EMD Biosciences). The resulting protein aggregates may then be utilized for the following purification method. The protein aggregates may be produced by the entity carrying out the purification method described herein. Alternatively, in certain embodiments, the protein aggregates may be derived from a third-party facility.

Before turning to the individual steps of the inventive purification method, it should be understood that all of the following steps of the protocol may be conducted at room temperature and atmospheric pressures, unless otherwise noted. Thus, each of the following steps (except the storage step at the end of the protocol) may occur at atmospheric pressure and at a temperature of at least 10, 15, 16, 17, 18, 19, or 20° C. and/or less than 30, 29, 28, 27, 26, or 25° C.

After providing the protein aggregate (as discussed above), at least a portion of the protein aggregate may then be at least partially solubilized in order to denature at least a portion of the protein in the protein aggregate. During the solubilization step, at least a portion of the protein aggregate may be contacted with at least one protein denaturant buffer and at least one pH buffer for at least one hour to thereby form a solubilized protein aggregate. This solubilization step causes the proteins in the protein aggregate to unfold and become linear. We have observed that the extended solubilization step (i.e., allowing the solubilization to occur for extended periods of time of greater than one hour and beyond) can result in a higher yield of purified prion protein amplification substrate, relative to existing protocols.

In one or more embodiments, the solubilization step can involve contacting at least a portion of the protein aggregate with at least one protein denaturant buffer and at least one pH buffer for at least 1, 2, 3, 4, 5, 6, 7, 8, 9, 10, 11, 12, 13, 14, or 15 hours and/or less than 36, 30, 24, or 18 hours. Furthermore, in various embodiments, the solubilization step may occur at a temperature of at least 10, 15, 16, 17, 18, 19, or 20° C. and/or less than 30, 29, 28, 27, 26, or 25° C. and at about atmospheric pressures. As noted above, it has been observed that these longer solubilization incubation times help increase the yield of the prion protein amplification substrate.

During the solubilization step, contact between the protein aggregate and the protein denaturant can be optimized and facilitated. For example, the solubilization step may occur under agitation so as to ensure repeated and intimate contact between the protein aggregate and protein denaturant. Thus, in certain embodiments, procedures that would mitigate the contact between the protein aggregate and the protein denaturant are preferably avoided during the solubilization step.

In one or more embodiments, at least 20, 21, 22, 23, 24, 25, 26, 27, 28, 29, 30, 31, 32, 33, 34, 35, 36, 37, 38, 39, or 40 mL of the protein denaturant buffer is added to the protein aggregate. Additionally, or in the alternative, less than 200, 150, 100, 75, 50, or 40 mL of the denaturant buffer is added to the protein aggregate.

The protein denaturant buffer can include any known protein denaturant buffer in the art, such as a detergent. In one or more embodiments, the protein denaturant buffer can be TRIS, SDS, urea, guanidine hydrochloride (e.g., 8 M guanidine hydrochloride), or a combination thereof.

The pH buffer generally has a pH in the range of 5 to 8, 5, to 7, 6 to 7, or about 6.5. Furthermore, the pH buffer can comprise any pH solvent capable of facilitating the protein denaturation process and modifying the pH of the mixture. In one or more embodiments, the pH buffer can comprise sodium phosphate (e.g., 100 mM NaPO₄).

In one or more embodiments, at least 0.5, 1, 2, 3, 4, or 5 mL of the pH buffer is added to the protein aggregate. Additionally, or in the alternative, less than 100, 50, 40, 30, 20, 10, 9, 8, 7, 6, or 5 mL of the pH buffer is added to the protein aggregate.

During the solubilization step, the protein denaturant buffer may be added at a rate of 5 to 40, 10 to 30, 15 to 25, 20 to 25, or about 20 mL of protein denaturant buffer per protein aggregate derived from one liter of bacterial expression. For example, if the protein aggregate is derived from one liter of bacterial expression, then 5 to 40 mL of protein denaturant buffer may be combined with the protein aggregate. In another example, if the protein aggregate is derived from two liters of bacterial expression, then 10 to 80 mL of protein denaturant may be combined with the protein aggregate.

The pH buffer may be added to the protein aggregate and protein denaturant mixture in an amount so as to optimize the pH of the mixture. The resulting solubilized protein aggregate may have a pH in the range of 5 to 8, 5, to 7, 6 to 7, or about 6.5.

After the solubilization step has been carried out for the desired amount of time, the resulting solubilized protein aggregate may be centrifuged so as to further purify the solubilized protein and remove the undesired residuals. In one or more bodies, the centrifugation may be carried out at about 12,000 rpm for 5 to 10to 50, 15 to 30, or about 15 minutes. The resulting supernatant from the centrifuged solubilized protein aggregate may then be utilized in the downstream purification steps, while the residual pellet is discarded.

Afterwards, at least a portion of the recovered supernatant may be contacted with at least one protein affinity resin to thereby form a resin mixture. During this step, at least a portion of the solubilized protein aggregate (e.g., the supernatant) may be contacted with at least one protein affinity resin under agitation and over a time period of at least 5, 10, 15, 20, 25, 30, 35, 40, or 45 minutes and/or less than 120, 110, 100, 90, 80, 70, 60, or 55 minutes to thereby form the resin mixture. During this step, at least a portion of the denatured protein in the solubilized protein aggregate may bind to least a portion of the protein affinity resin.

In one or more embodiments, the protein affinity resin can comprise Qiagen ni-nitrilotriacetic acid (NTA) superflux resin. Prior to contacting the solubilized protein aggregate, the protein affinity resin may be pretreated and prepared according to the manufacturer’s protocol (Qiagen). During this protocol, equal parts denaturation buffer (e.g., 6 M GDN buffer) may be combined with the protein affinity resin under agitation. Afterwards, the mixture is centrifuged and the supernatant is discarded.

In one or more embodiments, the resin mixture may comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mL of the solubilized protein aggregate. Additionally, or in the alternative, the resin mixture may comprise less than 90, 85, 80, 75, 70, 65, 60, 55, 50, 45, 40, 35, 30, 25, 20, 15, or 10 mL of the solubilized protein aggregate.

In one or more embodiments, the resin mixture may comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mL of the protein affinity resin. Additionally, or in the alternative, the resin mixture may comprise less than 90, 85, 80, 75, 70, 65, 60, 55, 50, 45, 40, 35, 30, 25, 20, 15, or 10 mL of the protein affinity resin.

Generally, the amount of protein affinity resin added to the solubilized protein aggregate depends on the bacterial expression used to form the protein aggregate. In one or more embodiments, at least 15, 16, 17, 18, 19, or 20 mL of the protein affinity resin is added per liter of bacterial expression.

In one or more embodiments, one or more of the following criteria can dictate the amount of protein affinity resin that is added to the resin mixture: (a) the resin mixture comprises less than 30 mL of the protein affinity resin, when the bacterial expression contains less than 13 grams of bacterial cells; (b) the resin mixture comprises at least 30 mL and less than 60 mL of the protein affinity resin, when the bacterial expression contains at least 13 grams and less than 20 grams of bacterial cells; and (c) the resin mixture comprises at least 60 mL of the protein affinity resin, when the bacterial expression contains at least 20 grams of bacterial cells. The amount of bacterial cells in the bacterial expression may be measured using techniques known in the art, such as via centrifugation and subsequent mass measurement (i.e., centrifuging the bacterial expression and weighing the formed pellet of cells).

Afterwards, at least a portion of the resin mixture may be introduced into a purification receptacle, such as a purification column. Alternatively, in various embodiments, the protein affinity resin and the solubilized protein may be combined and form the resin mixture in the purification receptacle. Exemplary cylindrical columns that may be used include the XK 16/70 Column, the XK 26/70 Column, and the XK 50/60 Column by Cytiva.

It has been discovered that it is very important to maintain a consistent height and width of the purification receptacle in order to ensure consistent concentration and contact between the protein affinity resin and solubilized protein in the receptacle. It has been observed that the height of the receptacle, particularly of a column, can be fairly critical and that one wants to maintain a relative consistent height of the receptacle when dealing with varying amounts of the resin mixture. In other words, and not wishing to be bound by theory, it has been observed that it is beneficial to modify the width or diameter of the purification receptacle, rather than the height, in order to optimize reaction conditions within the receptacle between the protein affinity resin and the solubilized protein. Thus, in many cases, the size and formulations of the initial bacterial expression and/or resin mixtures can dictate the desired size parameters of the purification receptacle.

Although not wishing to be bound by theory, it is believed that an increased diameter of the purification receptacle leads to a wider peak, thereby distributing the protein over a larger volume. It is important to keep the concentration from becoming too high so as to avoid self-aggregation, which leads to precipitation during dialysis and spontaneous conversion under normal assay conditions.

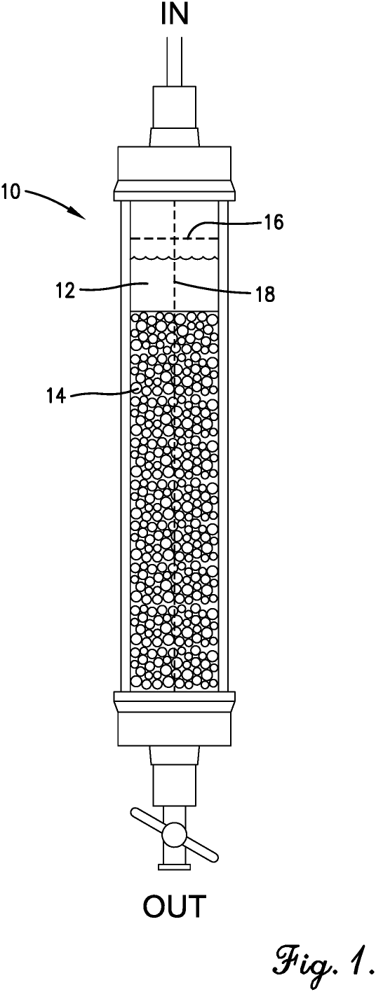

As shown in FIG. 1, the purification receptacle 10 may be at least partially filled with the resin mixture formed from the solubilized protein aggregate 12 and the protein affinity resin 14. Furthermore, as shown in FIG. 1, the purification receptacle has a maximum transverse interior dimension 16 and a maximum longitudinal interior dimension 18. In many cases, the maximum transverse interior dimension of the receptacle may refer to the diameter (if a column) or width (if a non-cylindrical shape) of the interior space within receptacle. Likewise, the maximum longitudinal interior dimension may refer to the height of the interior space within the receptacle.

In one or more embodiments, (i) the maximum transverse interior dimension is less than 25 mm and the ratio of the maximum longitudinal interior dimension to the maximum transverse interior dimension is at least 27:1, when the bacterial expression contains less than 13 grams of cells; (ii) the maximum transverse interior dimension is at least 25 mm and the ratio of the maximum longitudinal interior dimension to the maximum transverse interior dimension is less than 27:1 and greater than 12:1, when the bacterial expression contains at least 13 grams of cells and less than 20 grams of cells, and (iii) the maximum transverse interior dimension is at least 45 mm and the ratio of the maximum longitudinal interior dimension to the maximum transverse interior dimension is less than 15:1, when the bacterial expression contains at least 20 grams of cells.

The amount of protein affinity resin in the resin mixture introduced into the purification receptacle can also influence the required dimensions of the purification receptacle. In one or more embodiments, (i) the maximum transverse interior dimension is less than 25 mm and the ratio of the maximum longitudinal interior dimension to the maximum transverse interior dimension is at least 27:1, when the resin mixture introduced into the purification receptacle comprises less than 30 mL of the protein affinity resin; (ii) the maximum transverse interior dimension is at least 25 mm and the ratio of the maximum longitudinal interior dimension to the maximum transverse interior dimension is less than 27:1 and greater than 12:1, when the resin mixture introduced into the purification receptacle comprises at least 30 mL and less than 60 mL of the protein affinity resin, and (iii) the maximum transverse interior dimension is at least 45 mm and the ratio of the maximum longitudinal interior dimension to the maximum transverse interior dimension is less than 15:1, when the resin mixture introduced into the purification receptacle comprises at least 60 mL of the protein affinity resin.

The amount of solubilized protein aggregate in the resin mixture introduced into the purification receptacle can also influence the required dimensions of the purification receptacle. In one or more embodiments, (i) the maximum transverse interior dimension is less than 25 mm and the ratio of the maximum longitudinal interior dimension to the maximum transverse interior dimension is at least 27:1, when the resin mixture introduced into the purification receptacle comprises less than 30 mL of the solubilized protein aggregate; (ii) the maximum transverse interior dimension is at least 25 mm and the ratio of the maximum longitudinal interior dimension to the maximum transverse interior dimension is less than 27:1 and greater than 12:1, when the resin mixture introduced into the purification receptacle comprises at least 30 mL and less than 60 mL of the solubilized protein aggregate, and (iii) the maximum transverse interior dimension is at least 45 mm and the ratio of the maximum longitudinal interior dimension to the maximum transverse interior dimension is less than 15:1, when the resin mixture introduced into the purification receptacle comprises at least 60 mL of the solubilized protein aggregate.

In one or more embodiments, the maximum transverse interior dimension is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mm. Additionally, or in the alternative, the maximum transverse interior dimension is less than 60, 55, 50, 45, 40, 35, 30, 29, 28, 27, 26, 25, 24, 23, 22, 21, 20, 19, 18, 17, 16, 15, 14, 13, or 12 mm.

In one or more embodiments, the maximum longitudinal interior dimension is at least 100, 150, 200, 250, 300, 350, 400, 450, 500, 550, 600, 650, or 675 mm. Additionally, or in the alternative, the maximum longitudinal interior dimension is less than 1,000, 950, 900, 850, 800, 750, 700, 650, 600, or 550 mm.

In one or more embodiments, the ratio of the maximum longitudinal interior dimension to the maximum transverse interior dimension is at least 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, or 42:1. Additionally, or in the alternative, the ratio of the maximum longitudinal interior dimension to the maximum transverse interior dimension is less than 50:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, or 12:1.

In one or more embodiments, the purification receptacle has an average width of: (i) at least 12, 13, 14, 15, or 16 mm and/or less than 25, 24, 23, 22, 21, 20, 19, 18, or 17 mm, if the protein aggregate is derived from at least 0.8, 0.9, or 1.0 and/or less than 1.9, 1.8, 1.7, 1.6, 1.5, 1.4, 1.3, 1.2. or 1.1 liters of bacterial expression; and/or (ii) at least 20, 21, 22, 23, 24, 25, or 26 mm and/or less than 35, 34, 33, 32, 31, 30, 29, 28, 27, or 26 mm if the protein aggregate is derived from at least 1.6, 1.7, 1.8, 1.9, or 2.0 and/or less than 3.0, 2.8, 2.6, 2.5, 2.4, 2.3, or 2.2 liters of bacterial expression.

In one or more embodiments, the purification receptacle has an interior volume of at least 50, 55, 60, 65, 70, 75, 80, 85, 90, 95, 100, 105, 110, 115, 120, 125, 130, 135, 140, 145, 150, 175, 200, 225, 250, 275, 300, 325, 350, 375, 400, 425, 450, 475, 500, 550, 600, 650, 700, 750, 800, 850, 900, 950, or 1,000 cm³. Additionally, or in the alternative, the purification receptacle has an interior volume of less than 1,200, 1,150, 1,100, 1,050, 1,000, 950, 900, 850, 800, 750, 700, 650, 600, 550, 500, 450, 400, 375, 350, 325, 300, 275, 250, 225, 200, 175, or 150 cm³.

After being introduced into the purification receptacle, at least a portion of the resin mixture may be subjected to Fast Protein Liquid Chromatography (“FPLC”) to thereby form an eluted protein fraction and a spent affinity resin. During FPLC, a first pump and a second pump are used to subject at least a portion of the resin mixture to a gradient treatment starting with full contact with a denature buffer, followed by mixed treatments with a denature buffer and ending with treatments involving only the protein refold buffer.

The purification receptacle may be prepped for FPLC. Generally, the receptable may be prepped to have at least one denature buffer (e.g., 6 M guanadine hydrochloride, 100 mM NaPO₄, and 10 mM Tris at pH of 8.0) in A pump and at least one refold buffer (e.g., 100 mM NaPO₄ and 10 mM Tris at a pH of 8.0) in B pump. Subsequently, a gradient from 100% A to 100% B for 100 to 240 minutes, 140 to 200 minutes, or about 180 minutes at 0.4 to 1.0 mL/min, 0.6 to 0.9 mL/min, or about 0.8 mL/min may be carried out. At this point, the recombinant prion proteins are refolded and attached to the protein affinity resin. Afterwards, 100% B refold buffer (e.g., 100 mM NaPO₄ and 10 mM tris at a pH of 8.0) is pumped in until conductivity reaches 10 to 30 mS, 15 to 25 mS, or about 20 mS. Consequently, the recombinant prion proteins are all in the refold buffer and all the denature buffer from A has been removed.

Next, the receptacle is modified so as to have refold buffer (e.g., 100 mM NaPO₄ and 10 mM tris at a pH of 8.0) in A pump and the elution buffer (e.g., 100 mM NaPO₄, 10 mM Tris, and 500 mM imidazole) in B pump. Subsequently, the receptacle is reattached to the FPLC and a gradient is performed from 100% A to 100% B at 0.5 to 5.0 mL/min, 1.0 to 3.0 mL/min, or about 2.0 mL/min. Consequently, the recombinant prion proteins are eluted from the resin are now purified and are collected.

The resulting eluted protein fraction from the FPLC treatment may then be subjected to a dialysis treatment in order to further purify the proteins and form a dialyzed prion protein fraction. This step ensures that the protein will be functional and will not precipitate. During the dialysis treatment, at least one dialysis buffer is added to at least a portion of the eluted protein fraction so that the protein may be immediately diluted.

In one or more embodiments, at least 0.5, 1.0, 1.5, 2.0, 2.1, 2.2, 2.3, 2.4, 2.5, 2.6, 2.7, 2.8, 2.9, 3.0, 3.1, 3.2. 3.3, 3.4, 3.5, 3.6. 3.7, 3.8, or 3.9 mL of the dialysis buffer is added to the eluted protein fraction. Additionally, or in the alternative, less than 6.0, 5.5, 5.0, 4.5, 4.0, 3.5, 3.0, or 2.5 mL of the dialysis buffer is added to the eluted protein fraction.

In one or more embodiments, at least 3 mL of dialysis buffer is added to the eluted protein fraction, if the protein aggregate was derived from a bacterial expression with a volume of at least 2 and less than 3 liters. Additionally, in certain embodiments, at least 4 mL of dialysis buffer is added to the eluted protein fraction, if the protein aggregate was derived from a bacterial expression with a volume of at least 3 liters.

Although not wishing to be bound by theory, you generally want to increase the amount of dialysis buffer being used, along with the width or diameter of the purification receptacle, when a larger and more concentrated bacterial expression is utilized.

The amount of dialysis buffer utilized in the dialysis step may also influence the required dimensions of the purification receptacle. In one or more embodiments, (i) the maximum transverse interior dimension is less than 25 mm and the ratio of the maximum longitudinal interior dimension to the maximum transverse interior dimension is at least 27:1, when 2 mL or less of dialysis buffer is used; (ii) the maximum transverse interior dimension is at least 25 mm and the ratio of the maximum longitudinal interior dimension to the maximum transverse interior dimension is less than 27:1 and greater than 12:1, when greater than 2 mL and less than 4 mL of dialysis buffer is used, and (iii) the maximum transverse interior dimension is at least 45 mm and the ratio of the maximum longitudinal interior dimension to the maximum transverse interior dimension is less than 15:1, when greater than 4 mL of dialysis buffer is used.

The dialysis buffer can comprise any known buffer in the art. In one or more embodiments, the dialysis buffer comprises a phosphate buffer, TRIS, MOPS, HEPES, or a combination thereof. In various embodiments, the dialysis buffer comprises a 20 mM sodium phosphate with a pH of 5.5.

The dialysis treatment may occur at atmospheric pressure and at a temperature of at least 10, 15, 16, 17, 18, 19, or 20° C. and/or less than 30, 29, 28, 27, 26, or 25° C.

After the dialysis treatment, the protein concentration of at least a portion of the dialyzed prion protein fraction may be measured and adjusted using a spectrophotometer and additional dialysis buffer, which may be pre-filtered to remove any unwanted contaminants. This additional dialysis buffer can include any of the buffers noted above.

During this step, the dialyzed prion protein fraction may be diluted to a protein concentration of at least 0.30, 0.35, or 0.40 and/or less than 0.8, 0.75, 0.70, 0.65, 0.60, 0.55, 0.50, 0.45 mg/mL. In certain embodiments, the dialyzed prion protein fraction may be diluted to a protein concentration of about 0.45 mg/mL. It has been observed that failure to adequately dilute the protein at this stage may lead to undesirable aggregation and precipitation.

This dilution may occur at atmospheric pressure and at a temperature of at least 10, 15, 16, 17, 18, 19, or 20° C. and/or less than 30, 29, 28, 27, 26, or 25° C.

After diluting the dialyzed prion protein fraction to the desired concentration, at least a portion of the diluted protein fraction may then be filtered with one or more filters. For example, at least a portion of the diluted protein fraction may be filtered with a 0.22 µm filter syringe, followed by dialysis tubing with 0.22 µm and 33 mm filters.

The filtration may occur at atmospheric pressure and at a temperature of at least 10, 15, 16, 17, 18, 19, or 20° C. and/or less than 30, 29, 28, 27, 26, or 25° C.

After this filtration step, at least a portion of the filtered protein fraction may be subjected to further dialysis treatment in dialysis tubing for an extended period of time. For example, while in the dialysis tubing, at least a portion of the filtered protein fraction may be dialyzed in at least 1, 2, 3, or 4 liters and/or less than 10, 9, 8, 7, 6, or 5 liters of dialysis buffer for at least 0.1, 0.5, 1, 2, 3, or 4 hours and/or less than 24, 22, 20, 18, 16, 14, 12, 10, or 8 hours. It should be noted that this dialysis buffer can include any of the dialysis buffers noted above.

Subsequently, after this additional dialysis treatment is conducted, another dialysis treatment may be conducted. During this second dialysis treatment, while in the dialysis tubing, at least a portion of the filtered protein fraction may be again dialyzed in at least 1, 2, 3, or 4 liters and/or less than 10, 9, 8, 7, 6, or 5 liters of dialysis buffer for at least 0.1, 0.5, or 1 hours and/or less than 10, 9, 8, 7, 6, 5, 4, 3, or 2 hours. It should be noted that this dialysis buffer can include any of the dialysis buffers noted above.

It should be noted that either one of these dialysis treatments after the aforementioned filtration step may be optional and may be omitted as deemed necessary or desired.

After the dialysis treatments, the dialyzed protein fraction may then be subjected to additional filtration. During this filtration, at least a portion of the dialyzed protein fraction may be poured through one or more filters to thereby form a filtered dialyzed protein sample. In one or more embodiments, the filter may comprise a 0.22 µm filter, a 33 mm filter, or a combination thereof. In certain embodiments, this filtration step may be excluded if adequate filtration is utilized before dialysis.

The filtration may occur at atmospheric pressure and at a temperature of at least 10, 15, 16, 17, 18, 19, or 20° C. and/or less than 30, 29, 28, 27, 26, or 25° C.

After the filtration step, the protein concentration of the filtered dialyzed protein sample may then be optimized via dilution. As noted above, the protein concentration may be determined using a conventional spectrophotometer known in the art. During this step the filtered dialyzed protein sample may be diluted with one or more dialysis buffers, such as the ones listed above, to obtain a desired protein concentration in the purified product. It has been observed that one wants to avoid higher protein concentrations in the final product as this resulted in poorer protein substrate for RT-QuIC testing. Thus, an optimal protein concentration is desired in the purified protein product that would result in a protein amplification substrate ideally suited for RT-QuIC testing.

In one or more embodiments, the diluted protein sample has a final protein concentration of at least 0.2, 0.21, 0.22, 0.23, 0.24, 0.25, 0.26, 0.27, 0.28, or 0.29 and/or less than 0.45, 0.44, 0.43, 0.42, 0.41, 0.4, 0.39, 0.38, 0.37, 0.36, 0.35, 0.34, 0.33, 0.32, 0.31, or 0.3 mg/mL. In various embodiments, the diluted protein sample has a final protein concentration of 0.2 to 0.45 mg/mL.

The dilution may occur at atmospheric pressure and at a temperature of at least 10, 15, 16, 17, 18, 19, or 20° C. and/or less than 30, 29, 28, 27, 26, or 25° C. Furthermore, the dilution may be carried out under agitation in order to ensure mixing of the filtered sample and the additional dialysis buffer.

After the dilution treatment and obtaining the desired protein concentration, the diluted protein sample, which comprises refolded recombinant proteins, may then be stored for a set amount of time and temperature in order to yield the final purified protein amplification substrate. It has been observed that storage at temperatures above freezing conditions is preferable because this results in increased activity in the resulting purified protein amplification substrate, relative to purified protein amplification substrate that is immediately stored in freezing conditions (i.e., 0° C. and below). In addition, these higher storage temperatures also reduce shipping costs.

In one or more embodiments, at least a portion of the diluted protein sample is stored at a temperature of at least 1, 2, or 3° C. and/or less than 10, 9, 8, 7, 6, 5, or 4° C. for at least 1, 2, 4, 8, 12, 18, 24, 36, 48, 72, or 96 hours or at least 5, 10, 15, 20, 25, 30, 35, 40, 45, 50, 55, 60, 120, or 240 days.

In certain embodiments, at least a portion of the diluted protein sample is stored at a temperature greater than 0° C. and less than 10° C. for at least one hour to thereby form the purified protein amplification substrate.

In certain embodiments, at least a portion of the diluted protein sample is stored at a temperature of about 4° C. for at least one hour to thereby form the purified protein amplification substrate.

The purified protein amplification substrate formed by the protocol described herein may be used in any RT-QuIC testing procedures.

In one or more embodiments, an RT-QuIC kit may be provided that provides a prion protein amplification substrate, which can, optionally, be preloaded into a sample container and subsequently used for the amplification of the test prion for RT-QuIC testing. The protein amplification substrate may be from the same species as the source of the sample (e.g., whitetail deer) or may be from a different species relative to the sample source. Exemplary sources of the protein substrate can include bovine, ovine, hamster, rat, mouse, canine, feline, cervid, human, or non-human primate protein substrates.

Exemplary RT-QuIC testing procedures and further background information on purifying protein amplification substrates are described in PCT Pat. Application Publication WO 2022/016054; U.S. Pat. No. 8,216,788; U.S. Pat. Application Publication No. 2006/0263767; “Rapid End-Point Quantitation of Prion Seeding Activity with Sensitivity Comparable to Bioassay” by Wilham et al.; “Rapid Antemortem Detection of CWD Prions in Deer Saliva” by Henderson et al.; “Quantitative assessment of prion infectivity in tissues and body fluids by real-time quaking-induced conversion” by Henderson et al.; and “Longitudinal Detection of Prion Shedding in Saliva and Urine by Chronic Wasting Disease-Infected Deer by Real-Time Quaking-Induced Conversion” by Henderson et al.; the disclosures of which are incorporated herein by reference in their entireties.

### DEFINITIONS

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

As used herein, the term “inclusion bodies” and “inclusion body” refer to aggregates of specific types of proteins. The term “inclusion body” and “inclusion bodies” may be used interchangeably with the term “protein aggregate” herein.

### NUMERICAL RANGES

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

### CLAIMS NOT LIMITED TO DISCLOSED EMBODIMENTS

The preferred forms of the invention described above are to be used as illustration only, and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

What is claimed is:

1. A method for extracting and purifying a prion protein amplification substrate for RT-QuIC testing, the method comprising:
(a) providing a protein aggregate derived from an initial quantity of a bacterial expression;
(b) contacting at least a portion of the protein aggregate with at least 20 mL of at least one protein denaturant for at least one hour to thereby form a solubilized protein aggregate;
(c) contacting at least a portion of the solubilized protein aggregate with at least one protein affinity resin to thereby form a resin mixture;
(d) introducing at least a portion of the resin mixture into at least one purification receptacle, wherein the purification receptacle has a maximum transverse interior dimension and a maximum longitudinal interior dimension, wherein -
(i) the maximum transverse interior dimension is less than 25 mm and the ratio of the maximum longitudinal interior dimension to the maximum transverse interior dimension is at least 27:1, when the resin mixture introduced into the purification receptacle comprises less than 30 mL of the solubilized protein aggregate, and
(ii) the maximum transverse interior dimension is at least 25 mm and the ratio of the maximum longitudinal interior dimension to the maximum transverse interior dimension is less than 27:1, when the resin mixture introduced into the purification receptacle comprises at least 30 mL of the solubilized protein aggregate; and
(e) purifying at least a portion of the resin mixture to thereby form a purified protein amplification substrate.

2. The method according to claim 1, wherein the purified protein amplification substrate has a protein concentration of 0.2 to 0.45 mg/ml.

3. The method according to claim 1, wherein the purification receptacle is a column.

4. The method according to claim 1, wherein the resin mixture comprises at least 20 mL of the protein affinity resin.

5. The method according to claim 1, wherein the resin mixture comprises at least 15 mL of the protein affinity resin and at least 25 mL of the solubilized protein aggregate.

6. The method according to claim 1, wherein the purifying comprises subjecting at least a portion of the resin mixture to Fast Protein Liquid Chromatography (FPLC) to thereby form an eluted protein fraction and a spent affinity resin.

7. The method according to claim 6, wherein the purifying further comprises treating at least a portion of the eluted protein fraction with a dialysis buffer to thereby form a dialyzed prion protein fraction.

8. The method according to claim 7, wherein the treating comprises adding at least 2.5 mL of the dialysis buffer to at least a portion of the eluted protein fraction.

9. The method according to claim 7, wherein the purifying further comprises storing at least a portion of the dialyzed protein fraction at a temperature greater than 0° C. and less than 10° C. for at least one hour to thereby form the purified protein amplification substrate.

10. A method for extracting and purifying a prion protein amplification substrate for RT-QuIC testing, the method comprising:
(a) providing a protein aggregate derived from an initial quantity of a bacterial expression;
(b) contacting at least a portion of the protein aggregate with at least one protein denaturant for at least eight hours to thereby form a solubilized protein aggregate;
(c) contacting at least a portion of the solubilized protein aggregate with at least one protein affinity resin to thereby form a resin mixture;
(d) introducing at least a portion of the resin mixture into at least one purification receptacle;
(e) purifying at least a portion of the resin mixture to thereby form a purified protein amplification substrate; and
(f) storing at least a portion of the purified protein amplification substrate at a temperature greater than 0° C. and less than 10° C. for at least one hour to thereby form the prion protein amplification substrate.

11. The method according to claim 10, wherein the protein amplification substrate has a protein concentration of 0.2 to 0.45 mg/ml.

12. The method according to claim 10, wherein the purification receptacle is a column.

13. The method according to claim 10, wherein the purification receptacle has a maximum transverse interior dimension and a maximum longitudinal interior dimension, wherein -
(i) the maximum transverse interior dimension is less than 25 mm and the ratio of the maximum longitudinal interior dimension to the maximum transverse interior dimension is at least 27:1, when the resin mixture introduced into the purification receptacle comprises less than 30 mL of the solubilized protein aggregate, and
(ii) the maximum transverse interior dimension is at least 25 mm and the ratio of the maximum longitudinal interior dimension to the maximum transverse interior dimension is less than 27:1, when the resin mixture introduced into the purification receptacle comprises at least 30 mL of the solubilized protein aggregate.

14. The method according to claim 10, wherein the resin mixture comprises at least 15 mL of the protein affinity resin and at least 20 mL of the solubilized protein aggregate.

15. The method according to claim 10, wherein the purifying comprises subjecting at least a portion of the resin mixture to Fast Protein Liquid Chromatography (FPLC) to thereby form an eluted protein fraction and a spent affinity resin.

16. The method according to claim 15, wherein the purifying further comprises treating at least a portion of the eluted protein fraction with a dialysis buffer to thereby form a dialyzed prion protein fraction, wherein the treating comprises adding at least 2.5 mL of the dialysis buffer to at least a portion of the eluted protein fraction.

17. A method for extracting and purifying a prion protein amplification substrate for RT-QuIC testing, the method comprising:
(a) providing a protein aggregate derived from an initial quantity of a bacterial expression;
(b) contacting at least a portion of the protein aggregate with at least one protein denaturant for at least nine hours to thereby form a solubilized protein aggregate;
(c) contacting at least a portion of the solubilized protein aggregate with at least one protein affinity resin to thereby form a resin mixture;
(d) introducing at least a portion of the resin mixture into at least one purification column;
(e) subjecting at least a portion of the resin mixture to Fast Protein Liquid Chromatography (FPLC) to thereby form an eluted protein fraction and a spent affinity resin;
(f) contacting at least a portion of the eluted protein fraction with at least 2.5 mL of dialysis buffer to thereby form a dialyzed prion protein fraction; and
(g) storing at least a portion of the dialyzed protein fraction at a temperature of at least 1° C. and less than 10° C. for at least one hour to thereby form the purified protein amplification substrate.

18. The method according to claim 17, further comprising, subsequent to the contacting of step (f) and prior to the storing of step (g), diluting at least a portion of the dialyzed prion protein fraction to a protein concentration of 0.3 to 0.5 mg/mL.

19. The method according to claim 17, wherein at least 3.0 mL of the dialysis buffer is added to the eluted protein fraction.

20. The method according to claim 17, wherein the contacting of step (c) comprises adding at least 20 mL of the protein affinity resin.
